(19) United States
(12) Patent Application Publication
(10) Pub. No.: US 2005/0181050 A1
(43) Pub. Date: Aug. 18, 2005

(54) Dosage forms using drug-loaded ion exchange resins

(75) Inventors: Jane Hirsh, Wellesley, MA (US); Alison B. Fleming, Marshfield, MA (US); Roman Rariy, Allston, MA (US)

Correspondence Address: PATREA L. PABST;PABST PATENT GROUP LLP, 400 COLONY SQUARE, SUITE 1200, ATLANTA, GA, 30361, US

(73) Assignee: Collegium Pharmaceutical, Inc.

(21) Appl. No.: 11/046,608

(22) Filed: Jan. 28, 2005

Related U.S. Application Data
(60) Provisional application No. 60/539,677, filed on Jan. 28, 2004.

(51) Int. Cl. A61K 9/20; A61K 9/26; A61K 31/785

(52) U.S. Cl. 424/469; 424/78.1

(57) ABSTRACT

A multiparticulate, modified release composition for oral administration has been developed. The formulation is made by complexing a drug with an ion-exchange resin in the form of small particles, typically less than 150 microns. The present invention provides novel extended release coated ion exchange particles comprising drug-resin complexes, produced by binding the salt form of the drug, that do not require impregnating agents to insure the integrity of the extended release coat. To prepare a modified release formulation, one or more of the following types of particles are formulated into a final dosage form: (a) Immediate release particles, (b) Enteric coated particles, (c) Extended release particles, (d) Enteric coated-extended release particles; and (e) Delayed release particles. The various drug-containing particles described above can be further formulated into a number of different easy-to-swallow final dosage forms including, but not limited to, a liquid suspension, gel, chewable tablet, crushable tablet, rapidly dissolving tablet, or unit of use sachet or capsule for reconstitution

### CROSS-REFERENCE TO RELATED APPLICATION

This application claims priority to U.S. Ser. No. 60/539,677, entitled “Multiparticulate, Modified Release Drug Compositions for Oral Administration” by Jane Hirsh and Alison B. Fleming, filed Jan. 28, 2004.

### FIELD OF THE INVENTION

The present invention generally relates to improved dosage forms comprising drug loaded ion exchange resins.

### BACKGROUND OF THE INVENTION

Controlled or delayed release formulations are typically in solid form, consisting, for example, of a matrix system that releases drug over time via diffusion, an enteric coated tablet, or a polymer encapsulated drug which degrades and releases drug after a period of time. It is known in the art that solid oral dosage forms, such as tablets or capsules, are difficult for many patients to swallow. This is particularly true for pediatric and elderly patients as well as individuals that have difficulty swallowing (dysphagia) induced by disease states. One alternative for such patients is to crush tablets or other solid dosage forms and subsequently administer them within a liquid or semi-solid vehicle; however, crushing or splitting most extended or modified release solid dosage forms will result in an altered release profile and is thus a potentially dangerous practice.

Since conventional modified release tablets and capsules should not be crushed or manipulated, they are also not well suited when flexible dosing is required. This is particularly an issue at the outset of therapy when the dose of a drug is often incremented slowly up to an optimal level. Liquids are generally more amenable to dose titration of this nature. Unfortunately, few modified release liquids are available.

A few modified release liquids are available. Sustained release liquid suspensions comprising diffusion barrier coated, drug-loaded ion exchange resin particles are commercially available. The common method of production for such a composition involves several steps including: (1) loading of drug onto the ion exchange resin particles; (2) treating the drug-resin complex with a suitable impregnating agent; and (3) coating the resulting particles with an ethylcellulose coating using a solvent coating process (see U.S. Pat. No. 4,221,778). This process, although effective, involves the time consuming step of treatment with an impregnating agent as well as the costly and potentially hazardous step of coating from a solvent based solution.

What is needed is a more cost effective and safe method of production of diffusion barrier coated ion exchange resins.

It is therefore an object of the present invention to provide a novel preparation method for extended release particles based on coated ion-exchange resins.

It is a further object of the present invention to provide extended release coated ion exchange particles comprising drug-resin complexes, produced by that do not require impregnating agents to insure the integrity of the extended release coat.

It is a further object of the present invention to provide coated ion exchange resin compositions which provide modified release characteristics.

### SUMMARY OF THE INVENTION

An improved controlled release composition for oral administration has been developed. The formulation is made by complexing a drug with an ion-exchange resin in the form of small particles, typically less than about 150 microns. The resins are typically coated with one or more layers of coating material to provide a controlled pattern of release of drug from resin (“modified release”). To prepare a modified release formulation, one or more of the following types of particles are formulated into a final dosage form:

(a) Immediate release particles, which may be uncoated, coated with a polymer that dissolves in the oral cavity, that may also impart other properties such as mucoadhesion, or_coated with a polymer that is insoluble in the neutral medium of saliva, but dissolves in the acid environment of the stomach, that may impart other properties such as taste-masking;

(b) Enteric coated particles, prepared by coating drug-containing particles with a polymer that is insoluble in the acidic environment of the stomach but dissolves in the neutral environment of the small intestines;

(c) Extended release particles, prepared by coating drug-containing particles with a water insoluble but water permeable membrane;

(d) Enteric coated-extended release particles, prepared by coating extended release drug particles with a second enteric coating;

(e) Delayed release particles, prepared by coating drug-containing particles with a polymer that is insoluble in the acidic environment of the stomach and the environment of the mid to the upper small intestines, but dissolves in the lower small intestines or upper large intestines; and

(f) combinations thereof, either of two or more coatings on the same particles or formulations of particles having two or more different coatings.

The drug-loaded ion exchange resins for extended release drug delivery are coated from an aqueous dispersion of a synthetic polymer, most preferably poly(ethylacrylate-methylmethacrylate-triethylammonioethylmethacrylate chloride), available under the tradename Eudgragit RS 30 D. In some cases the complexation is carried out so that the final percentage by weight of the drug is below a critical threshold, which is approximately 30 to 35% by weight drug. Below this threshold, in contrast to previously reported results, the loaded particles may be coated without requiring impregnation with a volume-filling material to prevent rupturing of the coatings due to particle swelling.

These coated ion exchange resins can be further formulated into a number of different final dosage forms including, but not limited to, powder, liquid, liquid suspension, gel, capsule, soft gelatin capsule, tablet, chewable tablet, crushable tablet, rapidly dissolving tablet, and unit-of-use sachet or capsule for reconstitution.

### DETAILED DESCRIPTION OF THE INVENTION

The multiparticulate drug compositions described herein can have one or more types of release profiles. The multiparticulate drug compositions are obtained by complexing drug with a pharmaceutically acceptable ion-exchange resin and then coating the complexes with one or more polymeric coatings and/or mixing particles with two or more different polymeric coatings to form a single composition.

Definitions

Modified release dosage form: A modified release dosage form is one for which the drug release characteristics of time course and/or location are chosen to accomplish therapeutic or convenience objectives not offered by conventional dosage forms such as solutions, conventional ointments, or promptly dissolving dosage forms. Delayed release, extended release, and pulsatile release dosage forms and their combinations are the types of modified release dosage forms.

Delayed release dosage form: A delayed release dosage form is one that releases a drug (or drugs) at a time other than promptly after administration.

Extended release dosage form: An extended release dosage form is one that allows at least a twofold reduction in dosing frequency as compared to that drug presented as a conventional dosage form (e.g. as a solution or prompt drug-releasing, conventional solid dosage form).

Pulsatile release dosage form: A pulsatile release dosage form is one that mimics a multiple dosing profile without repeated administration and allows at least a twofold reduction in dosing frequency as compared to that drug presented as a conventional dosage form (e.g. as a solution or prompt drug-releasing, conventional solid dosage form). In one embodiment, a pulsatile formulation includes a mixture of particles releasing at different times, for example, a formulcation could contain equal amounts of immediate release particles and of enteric-coated extended release particles and thereby provide release in two pulses, immediate and after the drug particles reach the small intestine.

An immediate release dosage form is one that releases in the oral cavity or in the stomach. The immediate release dosage form may include a coating which imparts additional properties, such as a mucoadhesive coating enhancing uptake in the oral cavity, or a taste-masking coating that dissolves to release drug in the stomach.

As used herein the term “taste masking coating” refers to a pH dependent coating that is insoluble in the mouth but dissolves in the acidic pH of the stomach.

As used herein the term “extended release coating” refers to a pH independent substance that will act as a barrier to control the diffusion of the drug from its core complex into the gastrointestinal fluids.

As used herein, the term “enteric coating” refers to a coating material which remains substantially intact in the acid environment of the stomach, but which dissolves in the environment of the intestines.

As used herein the term “delayed release coating” refers to a pH dependent coating that is insoluble in the acidic pH of the stomach, the pH within the mid to the upper small intestine, but dissolves within the lower small intestine or upper large intestine.

As used herein, the term water-permeable is used to indicate that the fluids of the alimentary canal will permeate or penetrate the coating film with or without dissolving the film or parts of the film. Depending on the permeability or solubility of the chosen coating (polymer or polymer mixture) a lighter or heavier application of the coating is required to obtain the desired release rate.

I. Multiparticulate Drug Compositions

A. Drugs to Be Formulated

Exemplary drug agents useful for forming the composition described herein include, but are not limited to, analeptic agents; analgesic agents; anesthetic agents; antiasthmatic agents; antiarthritic agents; anticancer agents; anticholinergic agents; anticonvulsant agents; antidepressant agents; antidiabetic agents; antidiarrheal agents; antiemetic agents; antihelminthic agents; antihistamines; antihyperlipidemic agents; antihypertensive agents; anti-infective agents; anti-inflammatory agents; antimigraine agents; antineoplastic agents; antiparkinsonism drugs; antipruritic agents; antipsychotic agents; antipyretic agents; antispasmodic agents; antitubercular agents; antiulcer agents; antiviral agents; anxiolytic agents; appetite suppressants (anorexic agents); attention deficit disorder and attention deficit hyperactivity disorder drugs; cardiovascular agents including calcium channel blockers, antianginal agents, central nervous system (“CNS”) agents, beta-blockers and antiarrhythmic agents; central nervous system stimulants; diuretics; genetic materials; hormonolytics; hypnotics; hypoglycemic agents; immunosuppressive agents; muscle relaxants; narcotic antagonists; nicotine; nutritional agents; parasympatholytics; peptide drugs; psychostimulants; sedatives; sialagogues, steroids; smoking cessation agents; sympathomimetics; tranquilizers; vasodilators; beta-agonist; and tocolytic agents.

The drug is selected based on inclusion in the molecule of a group, such as an amino group, which will readily bind to a complexing agent such as an ion-exchange resin. Any drug that bears an acidic or a basic functional group, for example, an amine, imine, imidazoyl, guanidine, pyridinyl, quaternary ammonium, or other basic group, or a carboxylic, phosphoric, phenolic, sulfuric, sulfonic or other acidic group, can be bound to a resin of the opposite charge. Representative drug agents are described in, for example, WO 98/18610 by Van Lengerich, U.S. Pat. No. 6,512,950 and U.S. Pat. No. 4,996,047.

Some specific drugs that bear acidic or basic functional groups and thus may be complexed with an ion exchange resin include, but are not limited to Acetylsalicylic acid, Alendronic acid, Alosetron, Amantadine, Amlopidine,Anagrelide, Argatroban, Atomoxetine, Atrovastatin, Azithromycin dehydrate, Balsalazide, Bromocriptan, Bupropion, Candesartan, Carboplatin, Ceftriaxone, Clavulonic acid, Clindamycin, Cimetadine, Dehydrocholic (acid), Dexmethylphenidate, Diclofenac, Dicyclomine, Diflunisal, Diltiazem, Donepezil, Doxorubicin, Doxepin, Epirubicin, Etodolic acid, Ethacrynic acid, Fenoprofen, Fluoxetine, Furosemide, Gemfibrozil, Hydroxyzine, Ibuprofen, Imipramine, Levothyroxine, Maprolitline, Meclizine, Methadone, Methylphenidate, Minocycline, Mitoxantone, Moxifloxacin, Mycophenolic acid, Naproxen, Niflumic acid, Ofloxacin, Ondansetron, Pantoprazole, Paroxetine, Pergolide, Pramipexole, Phenytoin, Pravastain, Probenecid, Rabeprazole, Risedronic acid, Retinoic acid, Ropinirole, Selegiline, Sulindac, Tamsulosin, Telmisertan, Terbinafine, Theophyline, Tiludronic Acid, Tinzaparin, Ticarcillin, Valproic acid, Salicylic acid, Sevelamer, Ziprasidone, Zoledronic acid, Acetophenazine, Albuterol, Almotriptan, Amitriptyline, Amphetamine, Atracurium, Beclomethasone, Benztropine, Biperiden, Bosentan, Bromodiphenhydramine, Brompheniramine carbinoxamine, Caffeine, Capecitabine, Carbergoline, Cetirizine, Chlocylizine, Chlorpheniramine, Chlorphenoxamine, Chlorpromazine, Citalopram, Clavunate potassium, Ciprofloxacin, Clemastine, Clomiphene, Clonidine, Clopidogrel, Codeine, Cyclizine, Cyclobenzaprine, Cyproheptadine, Delavirdine, Diethylpropion, Divalproex, Desipramine, Dexmethylphenidate, Dexbrompheniramine, Dexchlopheniramine, Dexchlor, Dextroamphetamine, Dexedrine, Dextromethorphan, Diphemanil methylsulphate, Diphenhydramine, Dolasetron, Doxylamine, Enoxaparin, Ergotamine, Ertepenem, Eprosartan, Escitalopram, Esomeprazole, Fenoldopam, Fentanyl, Fexofenadine, Fluvastatin, Fluphenazine, Fluticasone, Fosinopril, Frovatriptan, Gabapentin, Galatamine, Gatifloxacin, Gemcitabine, Haloperidol, Hyalurondate, Hydrocodone, Hydroxychloroquine, Hyoscyamine, Imatinib, Imipenem, Ipatropin, Lisinopril, Leuprolide, Levopropoxyphene, Losartan, Mesalamine, Mepenzolate, Meperidine, Mephentermine, Mesalimine, Mesoridazine, Metaproteranol, Metformin, Methdialazine, Methscopolamine, Methysergide, Metoprolol, Metronidazole, Mibefradil, Montelukast, Morphine, Mometasone, Naratriptan, Nelfinavir, Nortriptylene, Noscapine, Nylindrin, Orphenadrine, Oseltamivir, Oxybutynin, Papaverine, Pentazocine, Phendimetrazine, Phentermine, Pioglitazone, Pilocarpine, Prochloroperazine, Pyrilamine, Quetapine, Ranitidine, Rivastigmine, Rosiglitazone, Salmetrol, Sertaline, Sotalol, Sumatriptan, Tazobactam, Tacrolimus, Tamoxifen, Ticlopidine, Topiramate, Tolterodine, Triptorelin, Triplennamine, Triprolidine, Tramadol, Trovofloxacin, Ursodiol, Promazine, Propoxyphene, Propanolol, Pseudoephedrine, Pyrilamine, Quinidine, Oxybate sodium, Sermorelin, Tacrolimus, Tegaseroid, Teriparatide, Tolterodine, Triptorelin pamoate, Scoplolamine, Venlafaxine, Zamivir, Aminocaproic acid, Aminosalicylic acid, Hydromorphone, Isosuprine, Levorphanol, Melhalan, Nalidixic acid, and Para-aminosalicylic acid.

Pharmaceutically acceptable salts of the above compounds may also be used.

B. Complexing Agents

Drug complexes are generally prepared by complexing the drug with a pharmaceutically acceptable ion-exchange resin. The complex is formed by reaction of a functional group of the drug with a functional group on the ion exchange resin. For example, a drug having a basic group such as an amino group can complex with an ion-exchange resin that bears an acidic group such as a sulfate or carboxylate group. Conversely, a drug that has an acidic group can complex with an ion-exchange resin that bears a basic group. Drug is released by exchanging with appropriately charged ions within the gastrointestinal tract.

Ion-exchange resins are water-insoluble, cross-linked polymers containing covalently bound salt forming groups in repeating positions on the polymer chain. The ion-exchange resins suitable for use in these preparations consist of a pharmacologically inert organic or inorganic matrix. The organic matrix may be synthetic (e.g., polymers or copolymers of acrylic acid, methacrylic acid, sulfonated styrene, sulfonated divinylbenzene), or partially synthetic (e.g., modified cellulose and dextrans). The matrix can also be inorganic, e.g., silica gel, or aluminosilicates, natively charged or modified by the addition of ionic groups. The covalently bound salt forming groups may be strongly acidic (e.g., sulfonic or sulfate acid groups), weakly acidic (e.g., carboxylic acid), strongly basic (e.g., quaternary ammonium), weakly basic (e.g., primary amine), or a combination of acidic and basic groups. Other types of charged groups can also be used, including any organic group that bears an acidic or a basic functional group, for example, an amine, imine, imidazoyl, guanidine, pyridinyl, quaternary ammonium, or other basic group, or a carboxylic, phosphoric, phenolic, sulfuric, sulfonic or other acidic group.

In general, those types of ion-exchangers suitable for use in ion-exchange chromatography and for such applications as deionization of water are suitable for use in these controlled release drug preparations. Such ion-exchangers are described by H. F. Walton in “Principles of Ion Exchange” (pp. 312-343) and “Techniques and Applications of Ion-Exchange Chromatography” (pp. 344-361) in Chromatography. (E. Heftmann, editor), Van Nostrand Reinhold Company, New York (1975). The ion-exchange resins typically have exchange capacities below about 6 meq./g. and preferably below about 5.5 meq./g.

Suitable resins include, but are not limited to, “Dowex” resins and others made by Dow Chemical; “Amberlite”, “Amberlyst” and other resins made by Rohm and Haas; “Indion” resins made by Ion Exchange, Ltd. (India), “Diaion” resins by Mitsubishi; Type AG and other resins by BioRad; “Sephadex” and “Sepharose” made by Amersham; resins by Lewatit, sold by Fluka; “Toyopearl” resins by Toyo Soda; “IONAC” and “Whatman” resins, sold by VWR; and “BakerBond” resins sold by J T Baker. Particular resins known to be useful include Amberlite IRP-69 (Rohm and Haas), and INDION 224, INDION 244, and INDION 254 (Ion Exchange (India) Ltd.). These resins are sulfonated polymers composed of polystyrene cross-linked with divinylbenzene.

The size of the ion-exchange particles should be less than about 2 millimeters, more preferably below about 1000 micron, more preferably below about 500 micron, and most preferably below about 150 micron (about 40 standard mesh). Commercially available ion-exchange resins (including Amberlite IRP-69, INDION 244 and INDION 254 and numerous other products) are typically available in several particle size ranges, and many have an available particle size range less than 150 microns.

As used herein, the term “regularly shaped particles” refer to those particles which substantially conform to geometric shapes such as spherical, elliptical, cylindrical and the like. As used herein, the term “irregularly shaped particles” refers to particles excluded from the above definition, such as those particles with amorphous shapes with increased surface areas due to surface area channels or distortions. For example, irregularly shaped ion-exchange resins of this type are exemplified by Amberlite IRP-69 (supplied by Rohm and Haas), and to the drug-resin complexes formed by binding drugs to these resins. Irregularly or regularly shaped particles may be used. The distinction between regularly shaped and irregularly shaped particles has been found by Kelleher et al (U.S. Pat. No. 4,996,047) to affect the degree of drug loading required to prevent swelling and rupture of extended release coatings when loaded resins are placed in aqueous solutions, in the absence of fillers or impregnating agents, such as polyethylene glycol. Kelleher, et al. found that the critical value was at least 38% drug (by weight in the drug/resin complex) in irregular resins, and at least 30% by weight in regular resins.

It has now been found that even if the loading of drug is less than the values described by Kelleher et al, it is still possible to make extended release coated resin particles that will not burst prematurely in aqueous suspension when not impregnated with glycerol or PEG or other agents preventing swelling of the resin. Thus, irregular ion exchange resins can be loaded with 36% of drug (by weight of the final composition, e.g., 36 g drug and 64 g ion exchange resin) and yet remain stable in an aqueous medium, particularly a nonionic aqueous medium A drug loading of 35%, 34%, 33%, 32%, 31%, 30%, or less than about 30%, can also be used with the irregularly shaped ion exchange drug resin particles. Similarly, regularly shaped ion exchange resin particles can be loaded with lower amounts of drug, such as 28%, 27%, 26%, 25% or 24% or less, to produce a non-swelling coated resin.

Ion exchange resins have pores of various sizes, which expand the area available for drug binding. The typical pore diameter is in the range of about 30 to 300 nanometers (nm), which is large enough for access by small-molecule drugs. For large drugs, such as proteins or nucleic acids, resins with larger pores, such as 500 to 2000 nm (0.5 to 2micron), often called “macroreticular” or “macroporous”, are preferred.

Binding of drug to resin can be accomplished according to four general reactions. In the case of a basic drug, these are: (a) resin (Na-form) plus drug (salt form); (b) resin (Na-form) plus drug (as free base); (c) resin (H-form) plus drug (salt form); and (d) resin (H-form) plus drug (as free base). All of these reactions except (d) have cationic by-products and these by-products, by competing with the cationic drug for binding sites on the resin, reduce the amount of drug bound at equilibrium. For basic drugs, stoichiometric binding of drug to resin is accomplished only through reaction (d).

Four analogous binding reactions can be carried out for binding an acidic drug to an anion exchange resin. These are: (a) resin (Cl-form) plus drug (salt form); (b) resin (Cl-form) plus drug (as free acid); (c) resin (as free base) plus drug (salt form); and (d) resin (as free base) plus drug (as free acid). All of these reactions except (d) have ionic by-products and the anions generated when the reactions occur compete with the anionic drug for binding sites on the resin with the result that reduced levels of drug are bound at equilibrium. For acidic drugs, stoichiometric binding of drug to resin is accomplished only through reaction (d).

Drug is bound to the resin by exposure of the resin to the drug in solution via a batch or continuous process (such as in a chromatographic colunm). Typically, the drug-resin complex thus formed is collected by filtration and washed with an appropriate solvent to insure removal of any unbound drug or by-products. The complexes are usually air-dried in trays. Such processes are described in, for example, U.S. Pat. Nos. 4,221,778, 4,894,239, and 4,996,047.

It has been found that incomplete loading of resin with drug can lead to a resin that does not require impregnation with an agent such as polyethylene glycol before coating. This allows the option of loading the drug onto the resin using the convenient salt: salt method described above, in which, for example, a soluble drug in a salt form is mixed with an ion exchange resin of opposite charge, also in a salt form. Alternatively, the non-salt forms of the drug and/or the resin can be used, with the total amount of drug to be applied kept at a lower level than that which would saturate the resin. In either case, the extra step of impregnation with polyethylene glycol or the like is eliminated.

C. Coatings
1. Immediate Release Coatings

Immediate release coatings are formed of a polymer that dissolves within the oral cavity upon contact with saliva or which are insoluble in the neutral pH of the oral cavity and which dissolve at the low pH of the stomach.

Coatings which dissolve in the mouth may have properties such as mucoadhesion, to prolong contact of the particles with the buccal, sublingual or other oral cavity surfaces to enhance uptake of the drug. Many mucoadhesive polymers are known and typically are characterized by a high density of carboxylic groups. See for example, U.S. Pat. Nos. 6,235,313 and U.S. Pat. No. 5,955,096 to Mathiowitz et al.

Coatings which dissolve in the stomach are typically used to provide properties such as taste-masking. Although binding drug to ion-exchange resins is a method of taste-masking known in the pharmaceutical art, unpleasant taste may be experienced when uncoated drug-resin complexes are orally administered. This may be a consequence of ion-exchange that occurs during the time that the drug-resin complexes are in the mouth, and may be a particular problem for chewable or rapidly dissolving solid formulations. Release of a bitter compound within the mouth makes such drug loaded ion-exchange resin particles unpalatable and irritating to the throat and esophagus. The coated particles of drug-resin complex prevent the release of drug within the mouth and insure that no unpleasant, bitter flavor is experienced by the patient consuming the dosage form.

The cationic polymer Eudragit® E 100 (Rohm Pharma) carries amino groups. Its films are, therefore, insoluble in the neutral medium of saliva, but dissolve by salt formation in the acid environment of the stomach. Such film coatings with a thickness of approximately 10 micrometers prevent medication with a bitter or revolting taste from dissolving in the mouth upon ingestion or during swallowing. The protective film dissolves quickly under the acidic conditions in the stomach allowing for the active ingredient to be released. A sugar coating may be used to accomplish similar taste-masking effect, albeit coating must be over 100 times thicker and the particles may result in tickling or irritating the throut.
2. Sustained or Extended Release Coatings

Extended release pharmaceutical compositions are obtained by complexing drug with a pharmaceutically acceptable ion-exchange resin and coating such complexes with a substance that will act as a barrier to control the diffusion of the drug from its core complex into the gastrointestinal fluids.

Control of the release of drugs from drug-resin complexes is possible with the use of a diffusion barrier coating on the drug-resin complex particles. Several processing methods to achieve extended release coatings on drug loaded resin particles have been described (see for example U.S. Pat. No. 4,996,047, 4,221,778, and 4,894,239); any of these may be used to obtain an extended release drug composition. The present invention discloses an alternative method of preparation of extended release coated drug-resin complexes without the use of impregnating agents.

U.S. Pat. No. 4,221,778 to Raghunathan describes the addition of solvating agents such as polyethylene glycol to the system in order to reduce the swelling of the drug-loaded resins and prevent the fracturing of the extended release coating. The solvating agent can be added as an ingredient in the resin drug complexation step or preferably, the particles can be treated with the solvating agent after complexing. This treatment has not only been found to help the particles retain their geometry, but has enabled the effective application of diffusion barrier coatings such as ethylcellulose to such particles. Other effective solvating (impregnating) agent candidates include, for example, propylene glycol, glycerin, mannitol, lactose and methylcellulose. Up to about 30 parts by weight (normally 10-25 parts) of the solvating agent to 100 parts by weight of the resin has been found to be effective. EP 171,528, EP 254,811, and EP 254,822 all disclose similar impregnation treatments in order to improve coatability of resin complexes.

Control of the release of drugs from drug-resin complexes has been achieved by the direct application of an ethylcellulose diffusion barrier coating to particles of such complexes in the absence of an impregnating agent, provided that the drug content of the complexes was above a critical value. U.S. Pat. No. 4,996,047 to Kelleher et al., discloses extended release coated drug-resin complexes wherein the drug comprises more than about 38% by weight (for irregularly shaped particles; over 30% for regular particles) of the dry drug-resin complex (based on the free acid or base of drug). In order to achieve this relatively high loading, a method of complexing drug to resin is provided whereby the drug is combined in its basic form with the resin in its acidic form (or visa versa). Since no ionic by-products are formed in such a reaction, very high loading levels are achieved. A similar scheme was disclosed in U.S. Pat. No. 4,894,239 to Nonomura, et al, with the free form of the drug being formed as part of a continuous process. U.S. Pat. No. 4,894,239 states that the drug-resin complex should contain at least 80% of the theoretical ion adsorption amount, and more preferably should contain about 85 to 100% of theoretical ion adsorption amount, to provide a stable ion exchange drug complex.

U.S. Pat. No. 5,186,930, Kogan et al. discloses drug-resin particles coated with a first inner coating of wax and a second outer coating of a polymer to achieve extended release. The inner wax coating prevents the swelling of the resins and subsequent rupturing of the extended release polymer coating.

In addition to known methods of processing drug-loaded resins to obtain stable extended release coatings, it was found that coating of drug loaded ion-exchange resins with an acrylic polymer based coating results in a stable extended release composition without use of impregnating agents, even when the drug loading is conducted by binding the salt form of the drug with the salt form of the resin, rather than binding the free base of the drug with resin in its acidic form as described by Kelleher et al and Nonomura et al. Drug-resin complexes obtained by binding the salt form of the drug with the salt form of the resin have drug loadings lower than Kelleher et al and Nonomura et al reported as necessary to obtain stable extended release coatings without the use of impregnating agents.

Any coating procedure which provides a continuous_coating on each particle of drug-resin complex without significant agglomeration of particles may be used. Coating procedures known in the pharmaceutical art including, but not limited to, fluid bed coating processes and microencapsulation may be used to obtain appropriate coatings. The coating materials of interest are copolymers available under the trade name Eudragit® (Rohm Pharma), such as poly(ethylacrylate-methylmethacrylate-triethylammonioethylmetharylate chloride) (Eudragit RS and Eudragit RL) and poly(ethylacrylate-methylmethacrylate) (Eudragit NE). Aqueous dispersions of such polymers are available under the trade names Eudragit RS 30 D, Eudragit RL 30 D and Eudragit NE 30 D. The preferred polymer for this purpose is a Eudragit RS.

These copolymers may be used singly, in admixture with each other, and in admixture with plasticizers (for example, triethyl citrate), pigments and other substances to alter the characteristics of the coating. In general, the major components of the coating should be insoluble in, and permeable to, water. However, it may be desirable to incorporate a water-soluble substance, such as methyl cellulose, to alter the permeability of the coating.

The coating materials are preferably applied as a suspension in an aqueous fluid. Drug loaded ion-exchange resins have typically been coated from solvent solutions (eg, U.S. Pat. Nos. 4,221,778, 4,894,239, and 4,996,047). Although coatings applied to ion-exchange resins are typically applied from solvent solutions, coating of ion exchange resins from aqueous dispersions has been described. U.S. patent application Ser. No. 2003/0099711 A1 describes coating ion exchange resins with an aqueous dispersion of ethylcellulose. The drug-resin complexes are treated prior to the coating process with an impregnating agent such as PEG. Ichikawa et al (International Journal of Pharmaceutics 216 (2001) 67-76) coated drug-loaded ion exchange resins with Eudragit RS 30 D. The loading of drug on the resin prior to coating was reported to be 56%. The coating level required to obtain sustained release was less than 6% but more than 2% by weight

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers are, but not limited to, polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent may be used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.
3. Enteric Coatings

In some embodiments drug-resin complexes are coated with a pH sensitive polymer which is insoluble in the acid environment of the stomach, and soluble in the more basic environment of the GI tract. Preventing drug release in the stomach has the advantage of reducing side effects associated with irritation of the gastric mucosa. Avoiding release within the stomach can be achieved using enteric coatings known in the art. The enteric coated formulation remains intact or substantially intact in the stomach, however, once the formulation reaches the small intestines, the enteric coating dissolves and exposes either drug-containing ion-exchange resin particles or drug-containing ion-exchange resin particles coated with extended release coating.

The enteric coated particles can be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et.al., (Media, PA: Williams and Wilkins, 1995). Examples of suitable coating materials include, but are not limited to, cellulose polymers, such as cellulose acetate phthalate, hydroxypropyl cellulose, hydroxypropyl methylcellulose phthalate and hydroxypropyl methylcellulose acetate succinate; polyvinyl acetate phthalate, acrylic acid polymers and copolymers, and certain methacrylic resins that are commercially available under the trade name Eudragit ® (Rohm Pharma). Additionally the coating material may contain conventional carriers such as plasticizers, pigments, colorants, glidants, stabilization agents, and surfactants.
4. Other Types of Delayed Release Coatings

In some embodiments drug-resin complexes are coated with a pH sensitive polymer which is insoluble in the acid environment of the stomach, insoluble in the environment of the small intestines, and soluble in the conditions within the mid to lower small intestine or upper large intestine (eg, above pH 7.0). Such a delayed release form is designed to prevent drug release in the upper part of the gastrointestinal (GI) tract.

The delayed release particles can be prepared by coating drug-containing microparticles with a selected coating material. Preferred coating materials are comprised of bioerodible, gradually hydrolyzable, gradually water-soluble, and/or enzymatically degradable polymers, and may be conventional “enteric” polymers. Delayed release polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit.RTM. (Rohm Pharma; Westerstadt, Germany), including Eudragit.RTM. L-100 (soluble at pH 6.0 and above), Eudragit.RTM. S (soluble at pH 7.0 and above, as a result of a higher degree of esterification), and Eudragits.RTM. NE, RL and RS (water-insoluble polymers having different degrees of permeability and expandability). Additional polymers include vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; and shellac. Combinations of different coating materials may also be used. Multi-layer coatings using different polymers may also be applied.

The preferred coating weights for particular coating materials may be readily determined by those skilled in the art by evaluating individual release profiles for drug loaded ion exchange resins with different quantities of various coating materials.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers are, but not limited to, polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Delayed release coated particles can be administered simultaneously with an immediate release dose of the drug. Such a combination produces the modified release profile referred to as “pulsatile release”. By “pulsatile” is meant that drug doses are released at spaced apart intervals of time. Generally, upon ingestion of the dosage form, release of the initial dose is substantially immediate, i.e., the first drug release “pulse” occurs within about one hour of ingestion. This initial pulse is followed by a first time interval (lag time) during which very little or no drug is released from the dosage form, after which a second dose is then released. Optionally, a second pulse is followed by a second time interval (lag time) during which very little or no drug is released from the dosage form, after which a third dose is then released.

The first pulse of the pulsatile release composition can be obtained by administering unmodified drug, uncoated drug-resin particles, immediate release particles (no coating, mucoadhesive or taste-masked coated drug-resin particles), or, in some cases, enteric coated drug-resin particles along with delayed release coated particles that provide a second pulse.

In some cases it may be advantageous to combine an immediately releasing dose of drug (eg, unmodified drug, uncoated drug-resin particles, or taste masking coated drug-resin particles) with enteric coated drug-resin particles to create a pulsatile profile. In this case the first pulse will occur substantially immediately and the second pulse will occur once the enteric coating has dissolved (in the upper small intestines).

In order to create a final dosage form with three pulses, an immediate release dose of drug (e.g., unmodified drug, uncoated drug-resin particles,mucoadhesive or taste masking coated drug-resin particles) can be combined with enteric coated drug-resin particles and delayed release coated drug resin particles.

In some cases where receptors are subject to saturation with a given drug, a distinct drop in plasma concentration may be required for optimal therapeutic performance. In these cases separating the first and second pulse of release by a significant time lag may be critical and may require the use of delayed release coated particles (rather than conventional enteric coated particles) in combination with an immediate release dose.

One of the advantages of a delayed release formulation is diminished incidence and reduced intensity of drug side effects, when compared to an immediate release form. A very common side effect that can be prevented is nausea. Other preventable side effects include vomiting, headache, tremulousness, anxiety, panic attacks, palpitations, urinary retention, orthostatic hypotension, diaphoresis, chest pain, rash, weight gain, back pain, constipation, vertigo, increased sweating, agitation, hot flushes, tremors, fatigue, somnolence, dyspepsia, dysoria, nervousness, dry mouth, abdominal pain, irritability, and insomnia.

II. Formulations Comprising Multiparticulate Drug Compositions

Formulations are prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients.

A. Liquid Suspension

Typically, the carrier in a liquid formulation will include water and/or ethanol, flavorings (bubblegum is a favorite for pediatric use) and colorings (red, orange, and purple are popular).

The coated drug-resin particles are suitable for suspending in an essentially aqueous vehicle with the only restrictions on its composition being (i) an absence of, or very low levels of ionic ingredients, and (ii) a limitation on the concentrations of water-miscible organic solvents, such as alcohol, and the pH to those levels which do not cause dissolution of the diffusion barrier and enteric coatings. Liquid oral dosage forms include aqueous and nonaqueous solutions, emulsions, suspensions, and solutions and/or suspensions reconstituted from non-effervescent granules, containing suitable solvents, emulsifying agents, suspending agents, diluents, sweeteners, coloring agents, and flavoring agents. Preservatives may or may not be added to the liquid oral dosage forms. Specific examples of pharmaceutically acceptable carriers and excipients that may be used to formulate oral dosage forms are described in U.S. Pat. No. 3,903,297 to Robert.

In preparing the liquid oral dosage forms, the drug-resin complexes are incorporated into an aqueous-based orally acceptable pharmaceutical carrier consistent with conventional pharmaceutical practices. An “aqueous-based orally acceptable pharmaceutical carrier” is one wherein the entire or predominant solvent content is water. Typical carriers include simple aqueous solutions, syrups, dispersions and suspensions, and aqueous based emulsions such as the oil-in-water type. The most preferred carrier is a suspension of the pharmaceutical composition in an aqueous vehicle containing a suitable suspending agent. Suitable suspending agents include Avicel RC-591 (a microcrystalline cellulose/ sodium carboxymethyl cellulose mixture available from FMC), and guar gum. Such suspending agents are well known to those skilled in the art.

Although water itself may make up the entire carrier, typical liquid formulations preferably contain a co-solvent, for example, propylene glycol, glycerin, sorbitol solution, to assist solubilization and incorporation of water-insoluble ingredients, such as flavoring oils into the composition.

B. Chewable, Crushable, or Rapidly Dissolving Tablets

In some embodiments, coated drug-resin complexes are incorporated into chewable tablets, crushable tablets, or tablets which dissolve rapidly within the mouth. Chewable tablet formulations containing coated particles are known in the pharmaceutical arts (see for instance the textbook “Pharmaceutical dosage form—tablets” Vol. 1 edited by H A Lieberman et al. Marcel Dekker, Inc. (1989)). Crushable tablets are the conventional tablets that have the same in vitro and in vivo performance regardless of their physical integrity, i.e. tablets can be crushed and administered as a powder, e.g. on apple sauce or mixed with water and syringed into a nasogastric or jujunostomy tube. The crushable tablets can be prepared using methods of tablet manufacturing known in the pharmaceutical art. Fast dissolving tablets containing coated particles are described, for example, in U.S. Pat. No. 6,596,311. These can also be administered as powders, for example, of antibiotics or other drugs which are dusted onto and/or into the area to be treated.

C. Gels

In some embodiments coated drug-resin complexes are incorporated into gels. Ion-exchange resin containing gel compositions are known in the art, see, for example, U.S. Pat. No. 4,837,255.

D. Reconstitutable Dosage Units

Coated drug-resin complexes can be formulated into a granular material and packaged in a sachet, capsule or other suitable packaging in unit dose. Such granular material can be reconstituted at the time of use into a suitable vehicle such as water. The granular material may contain excipients that facilitate the dispersion of the particles in water. Formulations of this type have been disclosed in U.S. Pat. No. 6,077,532.

E. Soft Gelatin Capsules

A soft gelatin capsule is a one piece sealed soft gelatin shell containing a liquid, a suspension, or a semisolid. Soft gelatin capsules can be filled with coated or uncoated drug-loaded particles, or mixtures thereof, suspended in a suitable solution. This can be an essentially non-ionic aqueous solution, or an emulsion. The incorporation of an ion exchange resin into a soft gelatin capsule provides a new versatility to this easy to swallow dosage form.

F. Optional Ingredients

Other optional ingredients well known to the pharmacist's art may also be included in amounts generally known for these ingredients, for example, natural or artificial sweeteners, flavoring agents, colorants and the like to provide a palatable and pleasant looking final product, antioxidants, for example, butylated hydroxy anisole or butylated hydroxy toluene, and preservatives, for example, methyl or propyl paraben or sodium benzoate, to prolong and enhance shelf life.

III. Combinations of Active Compounds

Multiple drugs may be simultaneously administered in the same dosage form. Acidic or basic drugs may be administered either as complexes with ion-exchange resins or as unbound compounds within the final formulation. These formulations may include, depending on the preparation, additional quantities of the same drug not absorbed to the resin, for example for achieving immediate release.

The other entities can also be other drugs, which can be on resins or coated while on resins; or may be present as particulates or in solution or dispersion, with or without coatings for control of release. Other entities could be instead, or in addition, be dissolved in a carrier or solvent, or, especially if liquid (but not exclusively), could comprise at least a portion of a carrier or solvent for the drug-loaded ion exchange resins, whether or not the latter are coated. The coating on the drug-containing ion-exchange particles may be an extended release coating, taste masking coating, enteric coating, delayed release coating or a combination of these coatings. If drug is in the formulation in an unbound form, drug particles can optionally be coated directly with the various coatings described above.

The drug-containing ion-exchange particles may be coated with an extended release coating, taste masking coating, enteric coating, delayed release coating or a combination of these coatings. If drug is in the formulation in an unbound form, drug particles can optionally be coated directly with the various coatings described above.

IV. Methods of Administration

The formulation can be administered to individuals in need thereof. Although preferred patients are human, typically any mammal including domestic animals such as dogs, cats and horses, may also be treated. The amount of the active ingredients to be administered is chosen based on the amount which provides the desired dose to the patient in need of such treatment to alleviate symptoms or treat a condition.

Exemplification

The present invention will be further understood by reference to the following non-limiting examples.

Drug-resin complexes were analyzed for drug content in the following manner: An accurately weighed, 90 mg sample (for uncoated complexes or coated complexes) was refluxed in 220 mL of an extraction solvent (10% 0.5M sodium acetate in ethanol) for 3 hours. Following refluxing, the mixture was cooled, transferred into a 250 mL volumetric flask with the aid of the extraction solvent, and the volume was brought up to 250 mL with extraction solvent. The resulting solution was analyzed for drug content via HPLC.

Coating was carried out in a fluidized bed coating apparatus, GPCG-1 (Glatt Air Techniques, Inc.).

Determinations of drug release from drug-resin complexes were performed with a Distek Dissolution Apparatus equipped with paddles rotating at 50 rpm. In all instances the release medium was maintained at 37° C. Samples obtained at various timepoints were analyzed via HPLC.

### Example 1

### Preparation of Dextromethorphan Loaded Ion-exchange Resins

Lot 1:

A. Loading of Dextromethorphan (HBr salt) to Amberlite IRP-69 (Na-form):

| Ingredient | Quantity/Batch |
|---|---|
| Dextromethorphan HBr, monohydrate | 600 g |
| Amberlite IRP-69, Na+ form | 1000 g |
| DI Water USP | qs |

Procedure:

Dextromethorphan was bound to ion exchange resin particles in a two-stage binding procedure. Briefly, Amberlite IRP-69 resin (1000 g) was added to deionized water (4.75 L) previously heated to 90° C. The resulting slurry was well mixed. Dextromethorphan HBr (300 g) was added to the resin slurry and subjected to mixing at 90° C. for 2 hours to allow binding to occur. The reaction slurry was then subjected to vacuum filtration in order to collect the resin particles. The resin particles were then washed with 10 L of pre-heated deionized water. The wet resin particles were re-suspended in 3 L of deionized water preheated to 90° C., and an additional 300 g of dextromethorphan HBr was added to the slurry while mixing. The second stage binding reaction was allowed to proceed for 2 hours. The reaction suspension was cooled to room temperature overnight. The reaction suspension was then filtered and washed three times with 10 L of pre-heated deionized water. The resulting drug-resin complex was dried in a forced draft oven at 45° C. until the loss on drying was less than 10% (as measured with a Mettler Toledo Moisture Analyzer at 110° C.).

The resulting dextromethorphan-resin complexes had the following properties:

| | | Drug Load |
|---|---|---|
| | | (% by weight of |
| | Loss on | dextromorphan base, |
| Lot # | Drying | on dry basis) |
| 1 | 6.5% | 31.1 |

B. Release of Dextromethorphan from Uncoated Complexes

Drug release was determined at 37° C. by adding drug-resin complex containing 30 mg equivalent of dextromethorphan HBr to 750 mL 0.1 N HCl in a dissolution vessel equipped with paddles rotating at 50 rpm. After 1 hour, the pH of the solution was changed to 6.8 in situ by the addition of 250 mL of 0.2 M tribasic sodium phosphate buffer. Samples were withdrawn periodically from the dissolution apparatus using an automated sampler and analyzed via HPLC.

The following release data was obtained, demonstrating that uncoated complex does not have significant extended release properties:

| | Lot 1 (Uncoated |
|---|---|
| | Dextromethorphan- |
| | Resin Complex) |
| Cumulative | % Dextromethorphan |
| Time (hrs) | Released |
| 0.1 N HCl | |
| 0.5 | 47 |
| 1 | 56 |
| pH 6.8 buffer | |
| 1.5 | 75 |
| 2 | 82 |
| 4 | 87 |
| 6 | 87 |
| 8 | 89 |
| 12 | 86 |

### EXAMPLE 2

### Preparation of Dextromethorphan Extended Release Ion Exchange Complexes

A. Preparation of Extended Release Coated Complexes

Lots 2,3 and 4:

Coating Composition:

| Ingredient | Quantity/Batch |
|---|---|
| Eudragit RS 30 D | 300 g |
| (Rohm Pharma Polymers) | |
| Triethyl Citrate FCC | 18 g |
| Talc USP | 45 g |
| DI Water USP | 402 g |
| Total | 765 g |

Coated drug-resin complexes were prepared by coating uncoated drug resin-complexes of Example 1 (Lot 1). A coating suspension was prepared by combining the ingredients in the table above. The suspension was filtered through a #100 mesh screen and kept under constant stirring during the coating procedure. Coating was carried out in a fluid bed coating apparatus equipped with a Wurster Column (GPCG-1, Glatt Air Techniques, Inc.). Samples were collected at three intervals in order to assess how the coating weight gain influenced release. Following coating, the product was well mixed with colloidal silicon dioxide at 1%. Finally, the coated particles were cured in a forced draft oven for 48 hours at 40° C. The conditions for the coating procedure were as follows:

Coating Parameters:

| Parameter | Value |
|---|---|
| Load of uncoated drug-resin complex | 525 g |
| Atomizing Air Pressure | 2.0 bar |
| Nozzle Size | 1.0 mm |
| Spray Rate | 5-7 g/min |
| Product Temperature | 21-25° C. |

B. Release of Dextromethorphan from Extended Release Coated Complexes

Drug release was determined at 37° C. by adding drug-resin complex containing 30 mg equivalent of dextromethorphan HBr to 750 mL 0.1 N HC1 in a dissolution vessel equipped with paddles rotating at 50 rpm. After 1 hour, the pH of the solution was changed to 6.8 in situ by the addition of 250 mL of 0.2 M tribasic sodium phosphate buffer. Samples were withdrawn periodically from the dissolution apparatus using an automated sampler and analyzed via HPLC.

| | Lot 2 (Low | | |
|---|---|---|---|
| | Coating Level) | Lot 3 (Medium | Lot 4 (High |
| | % Dextro- | Coating Level) % | Coating Level) % |
| Cumulative | methorphan | Dextromethorphan | Dextromethorphan |
| Time (hrs) | released | released | released |
| 0.1 N HCl | | | |
| 0.5 | 23 | 18 | 10 |
| 1 | 33 | 26 | 15 |
| pH 6.8 | | | |
| buffer | | | |
| 1.5 | 42 | 35 | 19 |
| 2 | 50 | 41 | 23 |
| 4 | 63 | 55 | 31 |
| 6 | 70 | 62 | 39 |
| 8 | 74 | 67 | 44 |
| 12 | 80 | 74 | 50 |

The coating level achieved on the drug-loaded ion exchange resin particles was estimated based on the drug content of uncoated versus coated resin particles. Lot 2 was found to be approximately 10.4% by weight coating; lot 3 was 12.9% by weight coating; and lot 4 was 16.2% by weight coating.

The release data demonstrates that the coating applied to the dextromethorphan-resin complexes is capable of controlling the release of drug.

EXAMPLE 3

### Preparation of an Extended Release Liquid Composition Containing Dextromethorphan Loaded Ion Exchange Particles

A. Preparation of a Suspension Containing Coated Resin Particles

Lot 5:

| Ingredient | Quantity/Batch | |
|---|---|---|
| DI Water | 415 | g |
| Tragacanth Powder, NF | 1.75 | g |
| Vanzan NF ED (Xanthan Gum) | 1.75 | g |
| Propylene Glycol, USP | 25 | g |
| Methyl Paraben, NF | 1 | g |
| Propyl Paraben, NF | 0.1 | g |
| Sucrose Crystal, NF | 50 | g |
| IsoClear 42 (High Fructose Corn Syrup) | 100 | g |
| FD& C Yellow # 6 | 0.005 | g |
| Citric Acid, Anhydrous, Granular, USP | 0.430 | g |
| Polysorbate 80 (Tween 80), NF | 0.035 | g |
| Dextromethorphan Particles, Coated, Lot 3 | 9.14 | g |

Tragacanth and xanthan gum were added to 250 g deionized water while mixing; mixing was continued for 20 minutes. High fructose corn syrup was then added and mixed for 5 minutes. Sucrose was added to 50 g deionized water, heated to boiling and allowed to cool to room temperature. Citric acid was dissolved directly into 75 g deionized water. The sucrose and citric acid solutions were then added to the bulk liquid and stirred for 5 minutes. Methylparaben and propylparaben were dissolved in propylene glycol, added to bulk liquid and mixed for 5 minutes. Yellow #6 was dissolved in 5 ml deionized water and mixed for 5 minutes. Polysorbate 80 was dissolved in 40 g deionized water, and the coated dextromethorphan-resin particles (Lot 3) were added to this solution and well mixed. The polysorbate/resin solution was then added to the bulk liquid and mixed slowly for 5 minutes.

B. Release of Dextromethorphan from Coated Resin in Suspension

Drug release was determined at 37° C. by adding 5 mL of suspension (containign 30 mg equivalent of dextromethorphan HBr) to 750 mL 0.1 N HC1 in a dissolution vessel equipped with paddles rotating at 50 rpm. After 1 hour, the pH of the solution was changed to 6.8 in situ by the addition of 250 mL of 0.2 M tribasic sodium phosphate buffer. Samples were withdrawn periodically from the dissolution apparatus using an automated sampler and analyzed via HPLC.

The following release data was obtained:

| Cumulative | Lot 5 (Coated Resin in Suspension) |
|---|---|
| Time (hrs) | % Dextromethorphan Released |
| 0.1 N HCl | |
| 0.5 | 17 |
| 1 | 26 |
| pH 6.8 buffer | |
| 1.5 | 38 |
| 2 | 46 |
| 4 | 63 |
| 6 | 71 |
| 8 | 75 |
| 12 | 81 |

The coated dextromethorphan-resin particles maintained their controlled release properties following formulation into an ion-free suspension for oral administration. For example, the suspension is suitable for treating persistent cough.

Modifications and variations of the compositions and methods of use thereof will be obvious to those skilled in the art and are intended to come within the scope of the following claims.

1. A drug formulation comprising
particles of a drug complexed to an ion exchange resin wherein the particles are coated with a polymeric coating selected from the group consisting of extended release coatings that maintain their integrity in an aqueous solution in the absence of an impregnating agent, delayed release coatings, immediate release coatings, and combinations thereof.

2. The formulation of claim 1 wherein the ion-exchange resin particles are less than about 150 microns in diameter.

3. The formulation of claim 1 wherein the coating is formed from an aqueous dispersion of a synthetic polymer.

4. The formulation of claim 3 wherein the coating is formed from an aqueous dispersion of a methacrylic ester co-polymer.

5. The formulation of claim 4 wherein the coating level is greater than 5% by weight.

6. The formulation of claim 1 wherein the coating is an extended release coating and the drug is present in an amount of less than about 35% by weight if the ion exchange resin is irregular in shape and less than about 28% if the ion exchange resin is regular in shape.

7. The formulation of claim 1 wherein the particles are taste-masked particles, prepared by coating drug particles with a polymer that is insoluble in the neutral environment of saliva, but dissolves in the acid environment of the stomach.

8. The formulation of claim 1 wherein the particles are coated with a polymer that is mucoadhesive in the oral cavity.

9. The formulation of claim 1 providing an extended release of drug to produce a therapeutic effect over approximately 24 hours.

10. The formulation of claim 1 providing an extended release of drug to produce a therapeutic effect over approximately 12 hours.

11. The formulation of claim 1 wherein the particles comprise less than about 50% by weight drug and an extended release coating on the drug-loaded ion exchange resin,
wherein the coating material is applied to the drug-resin particles from an aqueous dispersion.

12. The formulation of claim 1 comprising particles comprising an immediate release coating and a delayed release coating.

13. The formulation of claim 12 wherein the immediate release coating is a taste masking coating.

14. The formulation of claim 12 wherein the immediate release coating is a mucoadhesive coating.

15. The formulation of claim 1 comprising particles which have different coatings or wherein some particles are uncoated and some are coated.

16. The formulation of claim 15 providing pulsatile release.

17. The formulation of claim 1 wherein the delayed release coating is an enteric coating.

18. The formulation of claim 1 formulated into a dosage form selected from the group consisting of a gel, capsule, soft gelatin capsule, tablet, chewable tablet, crushable tablet, rapidly dissolving tablet, and unit of use sachet or capsule for reconstitution.

19. The formulation of claim 1 formulated into a liquid or liquid suspension.

20. The formulation of claim 1 wherein the drug is selected from the group consisting of analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti asthma drugs, cardiovascular drugs, corticosteroids, dopaminergics, electrolytes, gastro-intestinal drugs, muscle relaxants, nutritional agents, vitamins, parasympathomimetics, stimulants, anorectics, and anti-narcoleptics.

21. A method of administering a drug comprising administering the formulation of claim 1.

22. A method of making a drug delivery formulation comprising drug complexed to an ion exchange resin comprising
(i) binding drug to ion exchange resin particles;
(ii) coating the drug loaded resin particles in a fluid-bed coating apparatus, wherein the particles are coated with a polymeric coating selected from the group consisting of extended release coatings, delayed release coatings, immediate release coatings, and combinations thereof; and
(iii) formulating coated drug loaded resin particles into a final dosage form.

23. The method of claim 22 wherein the coating is sprayed from an aqueous dispersion of a synthetic polymer.

24. The method of claim 23 wherein the coating solution further comprises a plasticizer and a glidant.
